(19) United States
(12) Patent Application Publication
(10) Pub. No.: US 2009/0287218 A1
(43) Pub. Date: Nov. 19, 2009

(54) IMPLANT AND IMPLANTATION SYSTEM

(75) Inventors: Jens Beger, Tuttlingen (DE); Jörg Schumacher, Teltow (DE); Ralph Linke, Steisslingen (DE)

Correspondence Address: RATNERPRESTIA, P.O. BOX 980, VALLEY FORGE, PA, 19482, US

(73) Assignee: Aesculap AG, Tuttlingen (DE)

(21) Appl. No.: 12/437,966

(22) Filed: May 8, 2009

(30) Foreign Application Priority Data
May 13, 2008 (DE) 102008 024 440.6

(51) Int. Cl. A61B 17/58

(52) U.S. Cl. 606/94; 606/93

(57) ABSTRACT

An implant includes a tool receiver for coupling to an implantation tool and also a connecting device for connecting the implant to an injection cannula for the purposes of injecting a bone bonding material in such a manner that the handling and production thereof are simplified. The connecting device may include an internally threaded section which is formed in or on the tool receiver. An implantation system includes at least one injection cannula and at least one implant.

### CROSS-REFERENCE TO RELATED APPLICATIONS

This application claims priority to German Patent Application number 10 2008 024 440.6 filed May 13, 2008, the contents of which is incorporated herein by reference in its entirety and for all purposes.

### FIELD OF THE INVENTION

The present invention relates to an implant generally, and more specifically to an implant comprising a tool receiver for coupling to an implantation tool and also comprising a connecting device for connecting the implant to an injection cannula for the purposes of injecting a bone bonding material.

Furthermore, the present invention relates to an implantation system generally, and more specifically to an implantation system comprising at least one injection cannula for injecting a bone bonding material and at least one implant incorporating a tool receiver for coupling to an implantation tool and also a connecting device for connecting the implant to the at least one injection cannula.

### BACKGROUND

An implant of the type described hereinabove which is in the form of a bone screw is known from U.S. Pat. No. 6,048,343 for example. The known bone screw comprises a longitudinally extending channel having lateral openings in the distal end portion providing a fluidic connection to said channel. At the proximal end, a connecting device is provided on the head of the screw for connecting the implant to an adapter which enables the bone screw to be connected to a so-called Luer lock connector. A syringe can be attached to the adapter in order to convey adhesive under pressure through the channel of the bone screw to the outlet openings in the distal end thereof and then through said openings.

A disadvantage of the known bone screw lies in the type of connection which requires the adapter to be correctly placed on the head of the bone screw. Furthermore, the production process for the connecting device is expensive. Moreover, an adapter is needed in order to connect the bone screw to a syringe.

Therefore, it would be desirable to provide an implant and an implantation system of the type described hereinabove which can be easily handled and produced.

### SUMMARY OF THE INVENTION

In a first aspect of the invention an implant comprises a tool receiver for coupling to an implantation tool and also a connecting device for connecting the implant to an injection cannula for the purposes of injecting a bone bonding material. The connecting device comprises an internally threaded section which is formed in or on the tool receiver.

In a second aspect of the invention an implantation system comprises at least one injection cannula for injecting a bone bonding material and at least one implant incorporating a tool receiver for coupling to an implantation tool and also a connecting device for connecting the implant to the at least one injection cannula. The connecting device comprises an internally threaded section which is formed in or on the tool receiver.

### BRIEF DESCRIPTION OF THE DRAWINGS

The forgoing summary and the following description may be better understood in conjunction with the drawing figures, of which:

FIG. 1 shows a schematic perspective overall view of an implantation system;

FIG. 2 shows a sectional view along the line 2-2 in FIG. 1;

FIG. 2a shows an enlarged sectional view of a detail corresponding to FIG. 2 of an alternative embodiment of an implant system;

FIG. 3 shows a perspective view of a proximal end of an implant;

FIG. 4 shows a sectional view along the line 4-4 in FIG. 3;

FIG. 5 shows a sectional view along the line 5-5 in FIG. 3;

FIG. 6 shows a view similar to FIG. 3 of a further exemplary embodiment of an implant;

FIG. 7 shows a sectional view along the line 7-7 in FIG. 6; and

FIG. 8 shows a sectional view along the line 8-8 in FIG. 6.

### DETAILED DESCRIPTION OF PREFERRED EMBODIMENTS OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to an implant comprising a tool receiver for coupling to an implantation tool and also a connecting device for connecting the implant to an injection cannula for the purposes of injecting a bone bonding material, wherein the connecting device comprises an internally threaded section which is formed in or on the tool receiver.

Moreover, the present invention relates to an implantation system comprising at least one injection cannula for injecting a bone bonding material and at least one implant incorporating a tool receiver for coupling to an implantation tool and also a connecting device for connecting the implant to the at least one injection cannula, wherein the connecting device comprises an internally threaded section which is formed in or on the tool receiver.

The further development of a respective implant and an implant system of the type described hereinabove that is being proposed in accordance with the invention has the advantage that an injection cannula can be connected in a simple manner to the tool receiver which can be arranged at a proximal end of the implant for example. The provision of the internally threaded section makes it easy to tighten or screw a distal end of an injection cannula for example onto or into the implant. This enables the cannula to be removed even in a state of the bonding agent that is being injected, an adhesive or a bone cement for example, wherein the bonding agent has not yet fully hardened. In addition, this offers an optimal solution in those cases wherein there is only a small amount of clearance available for the connection of an injection cannula to the implant. Moreover, the proposed connecting device enables a particularly leak-proof connection for the bonding agent to be achieved. In addition, it can also be formed in such a manner as to nevertheless permit an exchange of gas to occur so that, when injecting a bonding agent, a gas-filled dead space in the cannula can escape and not be injected through the implant into the body of a patient. In each case, due to the provision of the internally threaded section in or on the tool receiver, it is ensured that, in contrast to systems in which the injection cannula is only clipped onto the implant, the tool receiver will not be contaminated. This makes it possible for the implant, a bone screw for example, to be tightened even after the process of injecting the fixing means into a part of the body into which the implant has been inserted since the tool receiver continues to be freely accessible. Furthermore, a dual function can thus be assigned to the tool receiver. On the one hand it can serve as a receiving means for a tool used for the insertion of the implant, whilst serving as part of the connecting device for connecting the implant to an injection cannula on the other.

The design of the implant is particularly simple, if the tool receiver is made in the form of a polygonal socket.

For example, the instruments and tools for inserting the implant that are available in an operating theatre can be used if the polygonal socket is made in the form of a square or a hexagonal socket.

Moreover, it can be advantageous if the tool receiver is in the form of a polygonal socket with inner rounded sides. Here too, there are standard tools available in an operating theatre for inserting implants into the body of a patient.

Advantageously, the polygonal socket with inner rounded sides is a hexagonal socket. Such a geometry enables the force being applied by the tool to be introduced into the tool receiver in an optimal manner.

Moreover, it is expedient if the tool receiver is in the form of an internal Torx®. Nowadays, Torx® tools of many different sizes are employed as standard devices for inserting implants.

In order to enable the implant to be connected to an injection cannula in a simple manner even after being at least partially inserted into the body of the patient, it is expedient if the connecting device is arranged at a proximal end of the implant.

The implant can be connected to an injection cannula in a simple manner, if it defines a longitudinal axis and if the tool receiver is formed such as to be coaxial with the longitudinal axis and is open in the proximal direction. This makes it possible for a user to at least partly insert a tool into the tool receiver even when a distal end of the implant has already been inserted into the body of a patient, for example, screwed into a bone.

Advantageously, the connecting device is formed in such a manner as to provide a cement-tight connection of the implant to the injection cannula. When the implant is connected to the injection cannula, this arrangement then ensures that bone bonding material cannot leak out in the general vicinity of the connecting device and thus contaminate the tool receiver in an unwanted manner.

Preferably, the connecting device is formed in such a manner that there is a gas-pervious connection of the implant to the injection cannula. This has the advantage in particular, that gas can escape from a dead volume of the injection cannula that is not filled with bone bonding material into the general vicinity of the connecting device thereby preventing it from being injected into the body of the patient. This can be disadvantageous especially in the case of implantation of the implant into a bone, since the whole of the area surrounding the implant could not then be filled with the bone bonding material in the desired manner. Consequently, a connection of the implant to the body of the patient can be significantly improved in each case. Moreover, the danger of an embolism is reduced.

A particularly positive and secure connection of the implant to the injection cannula can be achieved if the internally threaded section extends over the entire length of the tool receiver. Furthermore, this also has the advantage that contamination, even if just partial contamination, of the tool receiver with bone bonding material can be prevented.

The production of the implant is made particularly easy if the core diameter of the internally threaded section corresponds to a width across flats of the tool receiver or substantially to the width across flats. In this way, threaded sections are only worked into the inner surfaces of the tool receiver substantially in said surfaces themselves, but not necessarily over the entirety of those inner edges of the tool receiver which extend in the longitudinal direction. A secure and stable connection between a tool and the tool receiver can thereby be achieved without damaging the internally threaded section.

Particularly positive and secure engagement of the tool in the tool receiver can be achieved in the event that the outer diameter of the internally threaded section maximally corresponds to a corner diameter of the tool receiver. In this way in particular, the internal thread can be worked into the implant in a simple manner after the tool receiver has been formed.

Advantageously, the outer diameter of the internally threaded section maximally corresponds to 1.2 times the width across flats of the tool receiver. Due to this maximum ratio between the outer diameter and the width across flats, it is ensured that only as much material as necessary has to be removed from the implant in order to form the internal thread in or on the tool receiver.

Preferably, the outer diameter of the internally threaded section is smaller than a maximum external diameter of the tool receiver and greater than a minimum internal diameter of the tool receiver. In this way, it can be ensured that the depth of the threaded sections of the internally threaded section formed in the implant is deep enough for ensuring that there will be a secure connection between the injection cannula and the implant. Due to the fact that the diameter of the thread is smaller than a largest internal diameter or the maximum external diameter of the tool receiver, air gaps can then be formed through which any air remaining in the otherwise non-ventilated injection cannula can escape during the process of injecting the bone bonding material. By virtue of a correspondingly suitably small dimension of the gap however, any inadvertent leakage of cement can be prevented. The connection between the implant and the injection cannula thus additionally acquires a kind of filter function.

The implant can be anchored in the body of a person and in particular in a bone in a simple manner, if the implant is in the form of a bone screw incorporating a shank and a screw head.

The tool receiver on a bone screw is accessible in a simple manner, if it is formed on the screw head.

In order to enable the bone screw to be connected in a simple manner to other fixing elements with any arbitrary orientation, it is expedient if the screw head is substantially in the form of a ball head. Thus, in particular, poly-axial screws can be formed, i.e. screws which incorporate bearing elements having fixing seatings for rods or other parts for example, whereby the bearing elements are arranged on the screw head and are adjustable i.e. pivotal around a centre point of the ball head at least over a limited angular range.

In order to enable a bone bonding material to be injected after inserting the screw into a bone, it is advantageous if the implant comprises an injection channel and if there is provided at least one outlet opening which is in fluidic connection with the injection channel.

A bone bonding material can be introduced into the injection channel in a simple manner, if the tool receiver is in fluidic connection with the injection channel. This makes it possible for bone bonding material to be fed from the injection cannula, which is connected to the tool receiver, right through the tool receiver into the injection channel and then, optionally, out through one or more outlet openings of the injection channel. The outlet openings are preferably disposed in the vicinity of the distal end of the implant, but may be located along the entire length thereof. The possible cross-sectional shapes for the outlet openings are circles, squares or ovals as well as squares having rounded-off sides.

The bone bonding material can be fed through the implant and in particular to a distal end thereof in a particularly simple and secure manner, if the injection channel extends in the longitudinal direction of the implant. It preferably extends coaxially with respect to the longitudinal axis of the implant, and in the case of a bone screw, preferably coaxially with respect to a longitudinal axis defined by the shank of the bone screw.

In the case of the implantation system, it is expedient if the at least one injection cannula is made of a metal and/or a synthetic material. A simple and secure connection to the implant can be effected in this manner. Moreover, the stability of the injection cannula can also be ensured in a desired manner by appropriate choice of the material.

The injection cannula can be connected to the implant in a simple and secure manner, if the distal end of the at least one injection cannula has an external threaded section corresponding to the internally threaded section of the connecting device. The injection cannula can thereby be screwed into the internally threaded section in a simple and secure manner.

Advantageously, the proximal end of the at least one injection cannula is provided with a connecting element for the purposes of connection to an instrument for applying the bone bonding material. The connecting element makes it possible for the injection cannula to be connected to a suitable instrument for applying the bone bonding material in order to apply the pressure required for the application of the bone bonding material for example. In particular, the instrument for applying the bone bonding material can be manually operable or operable by means of a machine.

In order to enable routinely available standard instruments to be used for applying the bone bonding materials, it is expedient if the connecting element is in the form of a standard Luer connector. Connectors of this type, which are also referred to as a “Luer lock”, offer any desired combinations of injection cannulae and application instruments.

Moreover, it can be advantageous, if the implantation system also comprises a bone bonding material application instrument for applying a bone bonding material. The instrument for applying the bone bonding material thus forms a part of the implantation system.

In principle, it would be conceivable for the instrument for applying the bone bonding material to be empty or only suitable for forcing a bone bonding material contained in the injection cannula out of the injection cannula and into the implant. It is advantageous however, if the instrument for applying the bone bonding material is filled with bone bonding material. Expedient and suitable bone bonding materials are, in particular, bone cement or other biocompatible adhesives having a resin basis, such as methyl-methacrylate for example.

Moreover, it is advantageous if the at least one injection cannula forms a part of the instrument for applying the bone bonding material or if it can be connected to the instrument for applying the bone bonding material. In this way, it is possible to form the injection cannula both as an adapter for connecting the implant to the instrument for applying the bone bonding material or to connect the instrument for applying the bone bonding material directly to the implant.

Furthermore, it is expedient if the implant of the implantation system is one of the implants described above. The advantages given in connection with the preferred embodiments of the implant described then also apply to the implantation system as a whole.

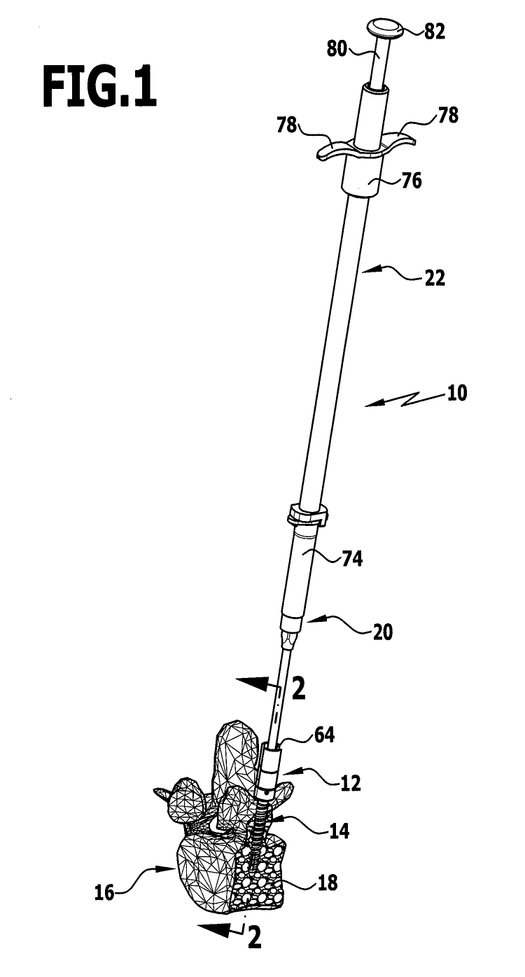

An implantation system bearing the general reference symbol 10 is illustrated in FIG. 1 by way of example. It serves for the implantation of an implant 12 in the form of a poly-axial bone screw 14 into a bone 16 of a human or animal body. In particular, the bone screw 14 may be a pedicle screw which can be screwed into the bone 16 that is in the form of a vertebra 18 of a spinal column, preferably, into a pedicle of the vertebra 18. Furthermore, the implantation system 10 comprises an injection cannula 20 and also an instrument for applying the bone bonding material 22.

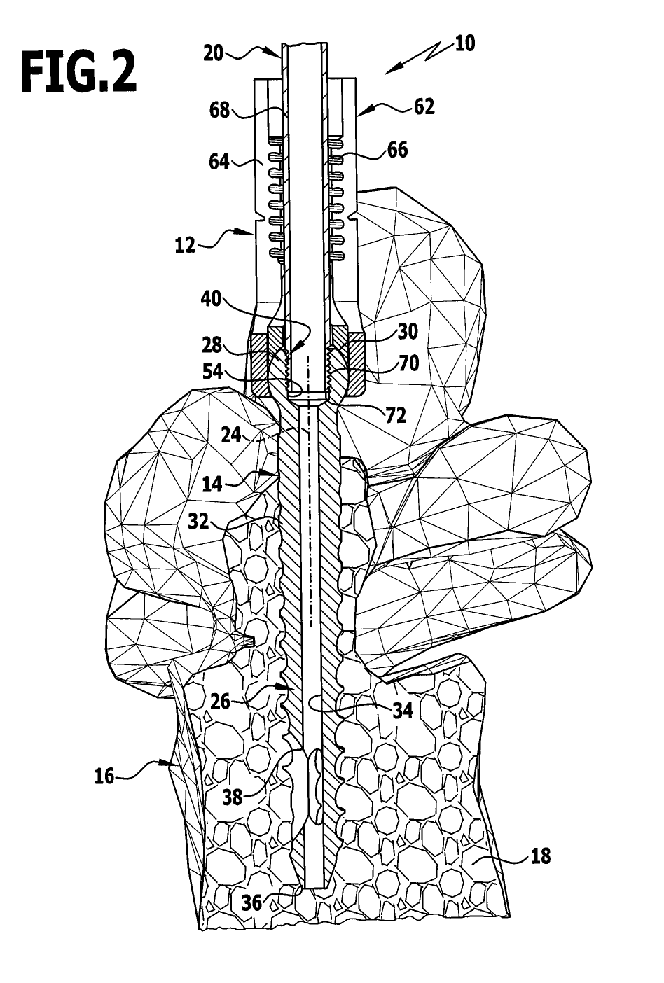

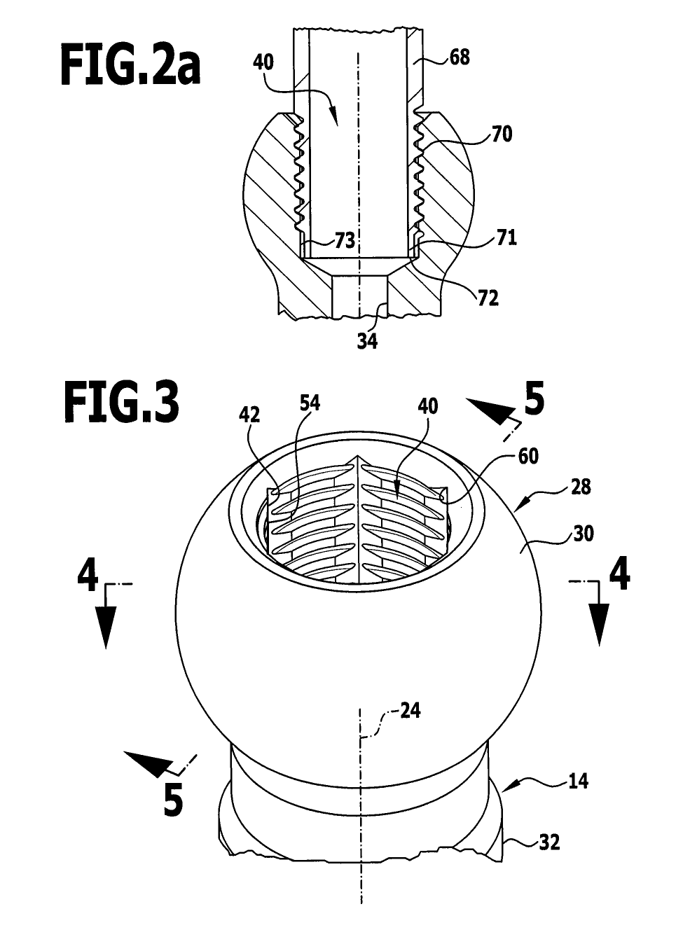

The bone screw 14 comprises a shank 26 which defines a longitudinal axis 24 and the proximal end thereof is provided with a screw head 28 in the form of a ball 30 having a maximum outer diameter that is somewhat greater than the maximum outer diameter of the shank 26. The shank 26 is provided over substantially the entire length thereof with an external thread 32 which is in the form of a bone thread the shape of which is optimised for the purposes of screwing it into the bone 16. The bone screw 14 contains a through boring that is coaxial with the longitudinal axis 34 so as to thereby form a cylindrical injection channel 34 which extends up to the distal end 36 of the bone screw 14. However, in contrast to the exemplary embodiment illustrated in FIGS. 1 and 2, the distal end of the bone screw 14 could also be closed.

For the special purpose of enabling a covering layer consisting of a bone bonding material, one consisting of bone cement for example, to be formed around the shank 26, two lateral outlet openings 38 in the form of longitudinally extending slits are provided in the shank 26. The outlet openings 38 are formed so as to be somewhat at the proximal side of the end 36. It is also conceivable however, for the outlet openings 38 to be of a different shape and for these to be optionally distributed over the entire length of the shank 26. The outlet openings 38 are in direct fluidic connection with the injection channel 34.

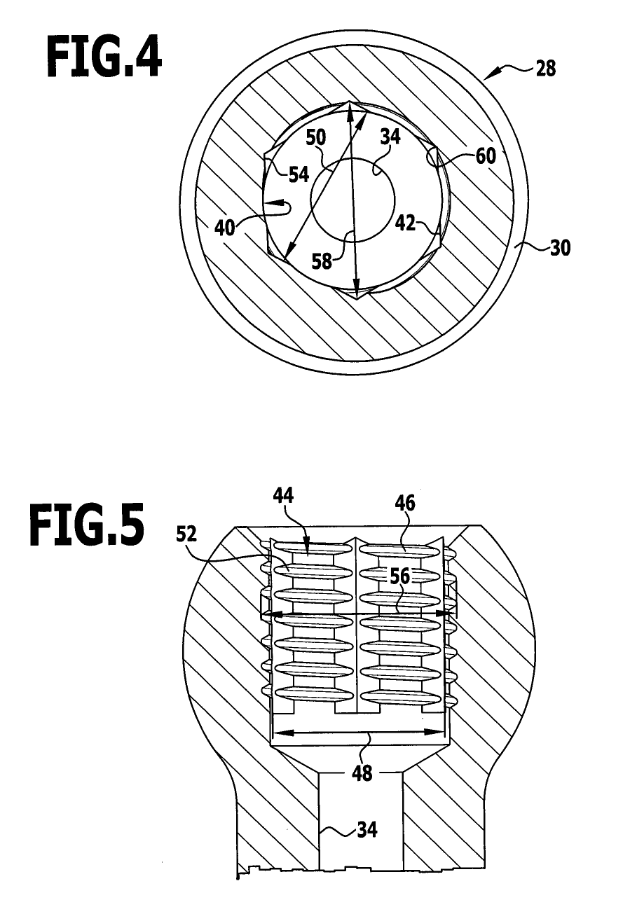

A tool receiver 40 extending coaxially relative to the longitudinal axis 24 is formed in the screw head 28, namely in the form of a polygonal socket 42 which takes the shape of a hexagonal socket in the arrangement illustrated in FIGS. 1 to 5. The tool receiver 40 is open in the proximal direction. Serving for the purposes of connecting the injection cannula 20 to the implant 12, there is a connecting device 44 which is formed in or on the tool receiver 40 and is in the form of an internally threaded section 46 that extends over practically the entire length of the tool receiver 40 in parallel with the longitudinal axis 24.

In the exemplary embodiment illustrated in FIGS. 1 to 5, the dimensions of the internally threaded section 46 are selected as follows: the core diameter 48 of the internally threaded section 46 substantially corresponds to the width across flats 50 of the polygonal socket 42. In this way, the flanks of the screw threads of the internally threaded section 46 are fully formed in the centre of those surfaces 54 of the polygonal socket facing in the direction of the longitudinal axis 24. Ideally, the diameter of the thread 56 of the internally threaded section 46 is selected to be smaller than or equal to a corner diameter 58 of the polygonal socket 42 and corresponds to approximately 1.2 times the width across flats 50. Consequently, the corners 60 of the hexagonal socket are still fully formed. They represent the regions at which the highest surface pressures arise during the torque transmitting process occurring when a not illustrated polygonal tool corresponding to the polygonal socket 42 is introduced into the tool receiver 40 and rotated about the longitudinal axis 24. A width across flats 50 may amount to 3.5 mm for example and the corresponding internally threaded section may be selected to be in the form of an M4×0.5 screw thread. The core diameter 48 then amounts to about 3.5 mm, and the corner diameter 58 to about 4.2 mm. In a further embodiment, the polygonal socket 42 may have a width across flats of 3.0 mm and the internally threaded section 46 may be an M3.5×0.5 screw thread. It is also conceivable for the polygonal socket to have a width across flats of 4.0 mm and the internally threaded section 46 to have a thread size of 4.5×0.5

A bearing sleeve 62 which comprises a seat corresponding to the screw head 28 and is pivotal about a centre point of the screw head 28 is placed on the screw head 28, said bearing sleeve comprising a slot 64 which extends transversely relative to the longitudinal axis 24 whereby a rod-shaped fixing element of a fixation system or a rod-shaped part of such a system can be inserted into said sleeve and fixed by means of a not illustrated screw. For this purpose, the bearing sleeve 62 has an internally threaded section 66. Optionally, the bearing sleeve 62 could also be rigidly connected to the screw head 28 so that, overall there is formed a mono-axial screw which is not illustrated in the Figures, i.e. the shank 26 and the bearing sleeve 62 which is also referred to as a so-called “tulip head” are then formed in one-piece.

The injection cannula is in the form of an elongated cylindrical sleeve 68 which comprises an externally threaded section 70 that commences from the distal end 72 and extends in the proximal direction and is formed in corresponding manner to the internally threaded section 46 so that the injection cannula 20 can be screwed onto the screw head 28. Optionally too, a short cylindrical section 71 which may optionally be conically tapering in the distal direction could also be formed from the distal end 72 as illustrated in FIG. 2a, the externally threaded section 70 being adjoined thereto at the proximal side thereof. The cylindrical section 71 projects into a short cylindrical section 73 of the tool receiver 40 which is formed on the distal side of the internally threaded section 46. An additional sealing effect can be achieved by optimally matching the dimensions of the cylindrical sections 71 and 73 so that bone bonding material cannot leak out into the transitional region between the end 72 and the injection channel 34.

In order to enable the injection cannula 20 to be guided through the bearing sleeve 42 up to the screw head 28, an outer diameter of the sleeve 68 is made somewhat smaller than a free internal diameter of the bearing sleeve 62. A length of the externally threaded section 70 parallel to the longitudinal axis 24 substantially corresponds to the length of the internally threaded section 46 so that the internally threaded section 46 and the externally threaded section 70 practically completely overlap when the injection cannula 20 is screwed onto the screw head 28.

Due to the selected threaded connection or due to the fact that the diameter of the thread 56 is smaller than the corner diameter 58, there are resultant air gaps through which an injection cannula 20 defining a dead volume can be evacuated of air, i.e. so that the air contained therein can thus escape therethrough.

At the proximal side thereof, the injection cannula 20 is equipped with an adapter 74 which can, in particular, be in the form of a Luer lock connector pointing in the proximal direction, this providing a standardised form of connection to the instrument for applying the bone bonding material 22. This is preferably in the form of a syringe which incorporates a cylindrical space for holding a bone bonding material and it also comprises a piston that is displaceably located therein. For the purposes of actuating the instrument for applying the bone bonding material 22, two laterally protruding wing-like projections 78 are formed on the cylinder 76 in the vicinity of the proximal end thereof and a disk-shaped head 82 is provided at the proximal end of the piston 80. By pressing on the head 82 with one's thumb, whereby for example the index finger and the middle finger of the same hand simultaneously grip under the projections 78 on the distal side thereof, the bone bonding material contained in the cylinder 76 can be forced into the injection cannula 20 and then through the latter into the injection channel 34 due to a movement of the piston 80 in the distal direction until such time as the bone bonding material emerges therefrom at the distal end and/or emerges laterally through the outlet openings 38 and forms a coating consisting of bone bonding material which surrounds the shank 26.

Due to the provision of the internally threaded section 46 in combination with the externally threaded section 70, the bone bonding material can be prevented from leaking out in the general vicinity of the tool receiver 40 and thereby contaminating it in such a manner that a tool element corresponding to the tool receiver 40 can no longer be introduced into the latter after the bone bonding material has been applied. Due to the special design of the internally threaded section 46 and the externally threaded section 70, air contained in the injection cannula 20 can escape as previously mentioned, but bone bonding material cannot leak out. There is thus provided a gas-pervious connection between the injection cannula 20 and the bone screw 14, but the connection is nevertheless cement-tight.

After the injection of the bone bonding material, the injection cannula 20 can still be removed in the pasty state of the material or during the initial part of a hardening process but without the screw head 28 being contaminated by the bone bonding material. It is also conceivable for a plurality of screws to be secured with the aid of the implantation system 10 by a process of injecting bone bonding material into the bone 16 wherein preferably each bone screw 14 is then connected to a separate injection cannula 20 and the instrument for applying the bone bonding material 22 is successively connected to the injection cannulae 20 and the bone bonding material 20 is injected.

The injection cannula 20 can, for example, be formed from a thin-walled metal tube or a tube of synthetic material. Furthermore, the instrument for applying the bone bonding material can be in the form of a standard syringe as illustrated in FIG. 1.

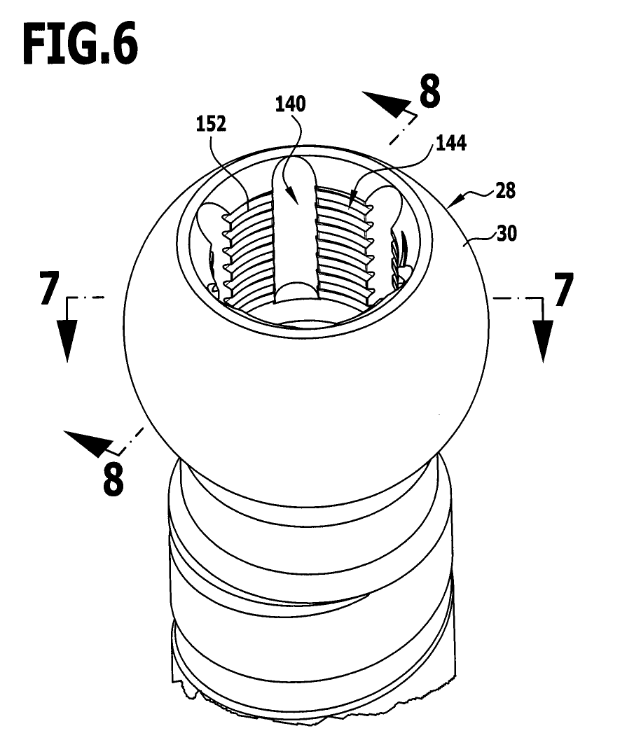

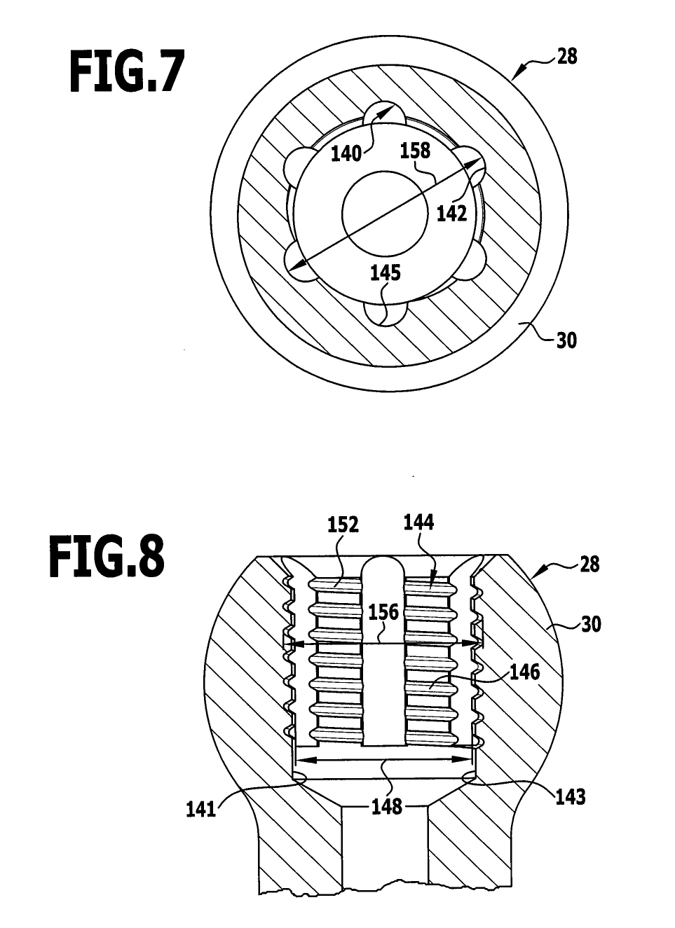

An alternative geometry for the tool receiver 40 is illustrated in FIGS. 6 to 8. In analogous manner to the tool receiver 40, a tool receiver 140 is formed on the screw head 28, namely, in the form of a polygonal socket 142 with inner rounded sides which has a simplified Torx® geometry. The tool receiver 140 is substantially in the form of a blind hole boring 141 having recesses 145 of substantially semicircular cross section worked into the inner wall 143 thereof, said recesses being evenly distributed around the periphery of the inner wall and extending in the longitudinal direction thereof. Corresponding semi-cylindrical projections which protrude from a cylindrical tool are adapted to engage in the recesses 145. An internally threaded section 146 extends approximately over the entire length of the tool receiver 140 and has a core diameter 148 which substantially corresponds to an inner diameter of the blind hole boring 141. The diameter of the thread 156 is somewhat greater than the internal diameter of the blind hole boring 141. The largest free diameter 158 between two mutually opposite recesses 145 is greater than the diameter of the thread 156. The flanks of the screw threads 152 are thus formed entirely in the inner wall 143 of the blind hole boring between the recesses 145 in this way.

The injection cannula 20 can also be screwed onto the screw head 28 in the case of the geometry illustrated in FIGS. 6 to 8. The connection resulting therefrom is also not gas-tight, because, when injecting the bone bonding material, a dead volume of air in the injection cannula 20 is not injected into the bone screw 14 and thus into the bone 16, but rather, it escapes in the connection region between the bone screw 14 and the cannula 22, and in particular, in the region of the recesses 45. The danger of embolism is thereby reduced. Nevertheless, the gaps thereby ensuing are small enough i.e. the order of magnitude thereof is such that the bone bonding material cannot leak out. Consequently, this connection too is gas permeable and cement-tight.

What is claimed is:

1. An implant comprising a tool receiver for coupling to an implantation tool and also a connecting device for connecting the implant to an injection cannula for the purposes of injecting a bone bonding material, wherein the connecting device comprises an internally threaded section which is formed in or on the tool receiver.

2. The implant in accordance with claim 1, wherein the tool receiver is in the form of a polygonal socket.

3. The implant in accordance with claim 2, wherein the polygonal socket is a square socket or a hexagonal socket.

4. The implant in accordance with claim 1, wherein the tool receiver is in the form of a polygonal socket with inner rounded sides.

5. The implant in accordance with claim 4, wherein the polygonal socket with inner rounded sides is a hexagonal socket.

6. The implant in accordance with claim 1, wherein the tool receiver is in the form of an internal Torx®.

7. The implant in accordance with claim 1, wherein the connecting device is arranged at a proximal end of the implant.

8. The implant in accordance with claim 1, wherein the implant defines a longitudinal axis and wherein the tool receiver is formed such as to be coaxial with the longitudinal axis and open in the proximal direction.

9. The implant in accordance with claim 1, wherein the connecting device is formed in such a manner that there is a cement-tight connection of the implant to the injection cannula.

10. The implant in accordance with claim 1, wherein the connecting device is formed in such a manner that there is a gas-pervious connection of the implant to the injection cannula.

11. The implant in accordance with claim 1, wherein the internally threaded section extends over the entire length of the tool receiver.

12. The implant in accordance with claim 1, wherein the core diameter of the internally threaded section corresponds to a width across flats of the tool receiver or substantially to the width across flats.

13. The implant in accordance with claim 1, wherein the outer diameter of the internally threaded section corresponds maximally to a corner diameter of the tool receiver.

14. The implant in accordance with claim 12, wherein the outer diameter of the internally threaded section corresponds maximally to 1.2 times the width across flats of the tool receiver.

15. The implant in accordance with claim 1, wherein the external diameter of the internally threaded section is smaller than a maximum external diameter of the tool receiver and greater than a minimum internal diameter of the tool receiver.

16. The implant in accordance with claim 1, wherein the implant is in the form of a bone screw comprising a shank and a screw head.

17. The implant in accordance with claim 16, wherein the tool receiver is formed on the screw head.

18. The implant in accordance with claim 16, wherein the screw head is in the form or substantially in the form of a ball head.

19. The implant in accordance with claim 1, wherein the implant comprises an injection channel and wherein there is provided at least one outlet which is in fluidic connection with the injection channel.

20. The implant in accordance with claim 19, wherein the tool receiver is in fluidic connection with the injection channel.

21. The implant in accordance with claim 19, wherein the injection channel extends in the longitudinal direction of the implant.

22. An implantation system comprising at least one injection cannula for injecting a bone bonding material and at least one implant incorporating a tool receiver for coupling to an implantation tool and also a connecting device for connecting the implant to the at least one injection cannula, wherein the connecting device comprises an internally threaded section which is formed in or on the tool receiver.

23. The implantation system in accordance with claim 22, wherein the at least one injection cannula is made of a metal and/or a synthetic material.

24. The implantation system in accordance with claim 22, wherein the at least one injection cannula comprises at its distal end an externally threaded section corresponding to the internally threaded section of the connecting device.

25. The implantation system in accordance with claim 22, wherein the at least one injection cannula comprises at its proximal end a connecting element for connection to an instrument for applying the bone bonding material.

26. The implantation system in accordance with claim 25, wherein the connecting element is in the form of a standard Luer connector.

27. The implantation system in accordance with claim 22, characterized by a bone bonding material application instrument for applying a bone bonding material.

28. The implantation system in accordance with claim 27, wherein the instrument for applying the bone bonding material is filled with a bone bonding material.

29. The implantation system in accordance with claim 27, wherein the at least one injection cannula forms a part of the instrument for applying the bone bonding material or can be connected to the instrument for applying the bone bonding material.

30. The implantation system in accordance with claim 22, wherein the implant defines a longitudinal axis and wherein the tool receiver is formed such as to be coaxial with the longitudinal axis and open in the proximal direction.

31. The implantation system in accordance with claim 22, wherein the connecting device is formed in such a manner that there is a cement-tight connection of the implant to the injection cannula.

32. The implantation system in accordance with claim 22, wherein the connecting device is formed in such a manner that there is a gas-pervious connection of the implant to the injection cannula.
